(19) United States
(12) Patent Application Publication
(10) Pub. No.: US 2021/0071229 A1
(43) Pub. Date: Mar. 11, 2021

(54) FIBRINOGEN TEST

(71) Applicant: DSM IP Assets B.V., Heerlen (NL)

(72) Inventors: Michael Johannes JANSSEN, Kaiseraugst (CH); San PUN, Kaiseraugst (CH)

(21) Appl. No.: 16/961,799

(22) PCT Filed: Jan. 25, 2019
(86) PCT No.: PCT/EP2019/051926

§ 371 Date: Jul. 13, 2020

(30) Foreign Application Priority Data
Jan. 25, 2018 (EP) 18153494.2

(51) Int. Cl. C12Q 1/56; C12Q 1/37

(52) U.S. Cl. CPC C12Q 1/56; G01N 2333/96422; G01N 2333/75; C12Q 1/37

(57) ABSTRACT

The present invention is related to a novel and direct method for measuring the fibrinogen level in a sample, which is particularly useful in emergency situations. The novel method is independent of thrombin formation and is not interfered by the presence of oral anti-coagulation drugs or other chemicals contrary to the commonly used clotting assays.

The present invention is related to a novel and direct method for measuring the fibrinogen level in a sample, which is particularly useful in emergency situations. The novel method is independent of thrombin formation and is not interfered by the presence of oral anti-coagulation drugs or other chemicals contrary to the commonly used clotting assays.

Fibrinogen is predominantly synthesized by the liver, with normal range between 1.5 to 3 mg/ml plasma. Fibrinogen is part of the clot formation occurring in bleeding disorders and thrombogenesis. Under normal conditions, fibrinogen-formation is activated by the action of thrombin (factor IIa), leading to cleavage of two short peptides, i.e. fibrinopeptide A and B, from the N-terminus of the alpha and beta polypeptide chains of fibrinogen. The newly formed N-terminal ends of the fibrin monomers spontaneously interact with the C-terminus of the fibrin monomers to form fibrin polymers, which under the influence of factor XIIIa, are crosslinked to form cross-linked fibrin polymers also known as clot formation.

Whereas in an event of massive blood loss thrombin or prothrombin are still able to sufficiently maintain the coagulation cascade—although on a very reduced level—fibrinogen is the first and key coagulation factor to reach critical levels. The quality of blood clots is heavily depending on fibrinogen concentration. Therefore, fibrinogen status is a key information upon emergency room admission. Instant and accurate fibrinogen level determination is paramount to trigger a medical strategy at pre-, peri- and post-operative stages, particularly in preparation of surgeries with high bleeding risks such as e.g. heart or liver surgeries. In case of fibrinogen levels below critical concentrations, the patient has to be supplied with fibrinogen concentrate and the like.

Currently, all available tests depend on the clotting cascade triggered at different levels which do not allow direct determination of the fibrinogen level in a sample. Upon addition of CaCl₂) to a sample such as blood or plasma the formation of clotting is measured. Their accuracy is greatly influenced or interfered by drugs, such as e.g. heparin, vitamin K-antagonists or direct or so-called novel oral anti-coagulants (DOACs or NOACs), which all aim on different parts of said clotting cascade. These interfering drugs can lead to false results which might cause high risk for the patient.

The standard nowadays is the so-called “Clauss-assay” (Mackie et al, Thromb Haemost. 2002 June; 87(6):997-1005) which is time-consuming and requires special buffer and thrombin reagent together with a high degree of technical expertise. Calibration is not straight forward. This method is based on a comparison between the clotting capability in a test sample with the clotting capability in a reference sample of derived fibrinogen concentration. The involvement of other factors, e.g. factor XIII, during clotting which cannot be ruled out in the quantification, variations of blood components either from endogenous or exogenous sources like drugs or hormones and/or variations in instruments and reagents from different suppliers making this method prone to interferences from many parameters. Hence, it is not possible to directly measure the fibrinogen level.

A further available method is the determination of the prothrombin time (PT), which is similar to the Clauss-assay in terms of endpoint measurement, i.e. the plasma fibrinogen level is defined indirectly by either optical measurement or mechanical strength measurement of factors involved in the clotting cascade. With this method a direct measurement of the fibrinogen level is also not possible. Compared to the Clauss-assay, the results of the PT are even more variable. Interference with DOACs/NOACs cannot be ruled out.

Another possible method is immuno-based ELISA, using the principle of antigen-antibody specific interaction. The ELISA technology is based on detection of concentrations in the range of ng per ml, i.e. requirement of strong sample dilution by a factor of million to reach the ELISA-compatible range, which is highly error-prone, tedious and introduces inaccuracy during the measurement. Furthermore, this test typically requires 4 hours of laboratory time, thus not applicable in emergency situations. Performance and/or interpretation of the test requires a lot of expertise by trained technicians.

Thus, it is an ongoing task to develop a more accurate, reliable, direct, platform-independent and fast method facilitating (daily) measurement of fibrinogen level in emergency situations but also in clinical laboratories including point-of-care testing (POCT), private home or in the typical working environment of veterinarians such as paddock or barn, leading to a quantitative determination of fibrinogen levels which works independently from the coagulation cascade and thus being not (negatively) influenced by anti-coagulation drugs or other interfering chemicals.

Surprisingly, we now developed such test method allowing direct and quantitative measurement of fibrinogen level in a sample, which is applicable in both humans or animals. The novel test method does not involve activation of the coagulation cascade and thus works independently on formation of thrombin, in contrast to the state-of-the-art test methods, e.g. Clauss-assay or the PT.

The novel test is based on enzyme kinetics, wherein the activity of a serine endopeptidase [EC 3.4.21], particularly snake venom serine endopeptidase, preferably venombin A [EC 3.4.21.74], is inversely proportional to the fibrinogen level in a given sample such as e.g. blood or plasma. The novel test works independently of blood coagulation, i.e. independently and without measurement of thrombin activity. Unlike the methods described in e.g. U.S. Pat. No. 4,692,496 or WO200136666 that are based on the activation of thrombin via CaCl₂) and wherein the clot formation is measured, the principle of the novel test method is based on an (intentional) inhibition of the blood coagulation cascade, e.g. of both intrinsic and extrinsic pathways. Thus, according to the present invention the fibrinogen level is measured via a change in enzymatic reaction speed of the serine-endopeptidase as defined herein, which is inversely proportional to an increase in fibrinogen concentration in the sample.

Thus, the present invention is directed to a novel method for measuring fibrinogen in a sample, such as e.g. blood or plasma, as well as to a diagnostic kit used for measuring the fibrinogen level in a sample, such as e.g. blood or plasma, said method being performed in the absence of CaCl₂) and/or in the absence of thrombin activity.

Particularly, the present invention is directed to a novel method for measuring fibrinogen in a sample, such as e.g. blood or plasma, as well as to a diagnostic kit used for measuring the fibrinogen level in such sample, said method being directly applied without the generation of a calibration curve and/or without the presence of any additional reference material, such as e.g. fibrinogen standards.

Furthermore, the present invention is directed to serine endopeptidase [EC 3.4.21], particularly snake venom serine endopeptidase, preferably venombin A [EC 3.4.21.74], used in a method for measuring the fibrinogen level in a sample, such as e.g. blood or plasma, as well as in a diagnostic kit used for such measurement.

Furthermore, the present invention is directed to a (detection) substrate, e.g. an artificial or natural (detection) substrate, preferably artificial (detection) substrate, used in a method for measuring the fibrinogen level in a sample, such as e.g. blood or plasma, as well as in a diagnostic kit used for such measurement, said method particularly comprising catalytic cleavage of said substrate by the serine endopeptidase [EC 3.4.21], particularly snake venom serine endopeptidase, preferably venombin A [EC 3.4.21.74].

Furthermore, the present invention is directed to a detectable moiety used in a method for measuring the fibrinogen level in a sample, such as e.g. blood or plasma, as well as in a diagnostic kit used for such measurement, as well as in a diagnostic kit used for such measurement, said method particularly comprising release of said detectable moiety by catalytic cleavage of the (detection) substrate via the action of said serine endopeptidase [EC 3.4.21], particularly snake venom serine endopeptidase, preferably venombin A [EC 3.4.21.74].

As used herein, the terms “level”, “status” or “concentration” in connection with fibrinogen are used interchangeably herein. The level of fibrinogen in a given sample, such as e.g. blood or plasma, is inversely proportional to the detected activity of the serine endopeptidase [EC 3.4.21].

The term “enzyme activity” as used herein refers to the proteolytic activity, i.e. cleavage of the (detection) substrate as defined herein resulting in release of a detectable moiety from the (detection) substrate which can be measured through methods known in the art and defined herein.

In one embodiment, the method/diagnostic kit as defined herein comprises protease inhibitors, such as e.g. inhibitors of fibrin polymerization leading to clot formation, particularly thrombin inhibitors, including but not limited to heparin.

A suitable serine endopeptidase [EC 3.4.21], particularly snake venom serine endopeptidase, preferably venombin A [EC 3.4.21.74], used in a method for measuring the fibrinogen level in a sample, such as e.g. blood or plasma, used for the performance of the present invention as well as in a diagnostic kit used for such measurement, might be selected from snake venom enzymes such as e.g. snake venom from Bothrops, Agkistrodon, Echis, Protobothrops, Calloselasma, Trimeresurus or Crotalus, preferably selected from B. moojeni, B. atrox, B. jararaca, E. pyramidum, E. carinatus, A. rhodostoma, P. mucrosquamatus, C. rhodostoma or C. adamanteus. More preferably, the enzyme is isolated from the venom of Bothrops moojeni (known as batroxobin) or Bothrops atrox, or an enzyme which is at least about 55%, such as at least about 60, 70, 80, 90, 95 or even 100% identical to batroxobin (such as UniProtKB—P04971), including, but not limited to enzymes known as calobin, ancrod (such as marketed under the tradename Viprinex®), flavoxobin, crotalase and further enzymes having serine endopeptidase activity as defined herein.

A suitable (detection) substrate to be used for the performance of the present invention including a diagnostic kit used for such measurement might be selected from any artificial or natural (detection) substrate, particularly artificial substrate, which can be catalytically cleaved via action of the serine endopeptidase as defined herein, leading to release of a detectable moiety from the substrate as defined herein.

Thus, in one embodiment the present invention is directed to a (detection) substrate linked to a detectable moiety used in a method for measuring the fibrinogen level in a sample, such as e.g. blood or plasma as well as in a diagnostic kit used for such measurement.

The detection method includes but is not limited to mechanical (non-clot based), amperometric (electrochemical), optical, electromechanical, photoelectrochemical, electrogenic or photo-mechanical detection which are currently used in POC-devises or laboratory testing. The detection method/technology is based on the detection/measurement of the released and detectable moiety by said enzymatic cleavage of said serine endopeptidase from said (detection) substrate, particularly artificial substrate, linked to the detectable moiety. The release of the detectable moiety from the (detection) substrate, particularly artificial substrate, produces a change in detectable/measurable signals for the appropriate method/technology. Particularly, the detectable moiety can be detected/measured through methods of (photo)electrochemical, amperogenic, chromogenic and/or fluorogenic principles.

In one embodiment, the artificial substrates include but are not limited to substrates according to formula (I) to (X) in Table 1, such as e.g. known under the tradename Pefachrome®TH, Electrozyme TH, H-D-phenylalanyl-pipecolyl-arginine-p-amino-p-methoxydiphenylamine (PPAAM), toluolsulfonyl-glycyl-prolinyl-arginin-4-amido-2-chlorophenol or substrates according to WO2009053834 (e.g. paragraph [0047]), WO2000050446 (e.g. page 5 to 6) or WO2016049506 (e.g. paragraph [0018] and [0019]) or substrates of formula (I) to (X) but with alternative protecting groups at the N-terminal part and/or substrates of formula (I) to (X) with additional amino acids introduced between the protecting group and the 1^{st} N-terminal amino acid shown in formula (I) to (X). The skilled person knows which protecting groups to use, e.g. butyloxycarbonyl, formyl and the like.

TABLE 1
List of putative (detection) substrates. pNA = p-nitroanilide.
But = L-α-aminobutyric acid (2-aminobutyric acid).
Pip = L-pipecolic acid. AMC = 7-amido-4-methylcoumarin.
For more details, see text.

| Chemical formula | Formula # |
|---|---|
| Tos-Gly-Pro-Arg-pNA AcOH | (I) |
| H-D-CHG-Ala-Arg-pNA·2AcOH | (II) |
| H-D-CHG-But-Arg-pNA·2AcOH | (III) |
| H-D-CHA-Ala-Arg-pNA·2AcOH | (IV) |
| H-D-CHA-Gly-Arg-pNA·2AcOH | (V) |
| CH₃OCO-Gly-Pro-Arg-pNA·AcOH | (VI) |
| H-ß-Ala-Gly-Arg-pNA·2AcOH | (VII) |
| H-D-Phe-Pip-Arg-pNA·2HCl | (VIII) |
| H-D-CHA-Ala-Arg-AMC·2AcOH | (IX) |
| BZ-L-Phe-L-Val-L-Arg-pNA | (X) |

The method as described herein for measuring the fibrinogen level in a sample as well as a diagnostic kit used for such measurement includes the detection of proteolytic activity of an enzyme as defined herein, said method can be performed on any device suitable for detection of such proteolytic activity, such as e.g. Xprecia Stride (Siemens Healthcare), CoaguChek® (Roche Diagnostics), i-Stat® systems (Axonlab/Abbott), ESR or qLabs systems (Operon Biotech a Healthcare), Alere INRatio® systems (Alere™), LabPad® (Avalun®), microINR (iLine® Microsystems), Mission® PT (Acon®) or other systems used or known in the art for lab-based tests or POCT in the field of blood analysis.

The method for measuring the fibrinogen level in a sample as defined herein as well as a diagnostic kit used for such measurement comprises measuring the proteolytic activity of a serine endopeptidase as defined herein, such as e.g. batroxobin, on a defined artificial and/or natural substrate, particularly artificial substrate, which is linked to a particular detectable moiety as defined herein, i.e. any chemical or particle group that facilitates detection of proteolytic activities, such as e.g. a fluorogenic, chromogenic, amperogenic and/or (photo)chemical group linked to the detection substrate. The proteolytic activity is measured in relationship to time, i.e. the speed of signal generation due to proteolytic activity, wherein the detected signal indicates the catalytic cleavage activity of the artificial and/or natural, particularly artificial, substrate detectable by different technologies known in the art. This speed is influenced by the presence of fibrinogen. The proteolytic release of a detectably moiety as defined herein, such as pNA, can be measured at specific OD, such as e.g. OD405. The more pNA being released, the higher OD405, the faster the enzyme, such as e.g. batroxobin works, the less fibrinogen is present in the reaction.

As used herein, measurement “OD405” means measurement of the optical density (OD) at 405 nm of light. The released pNA gives color to the reaction, which can be measured at the maximal absorption (which is 405 nm).

Thus, a high level of fibrinogen in a sample, i.e. a level of at least about 5 mg/ml sample, results in a least steep curve (indicating less detection-substrate to be cleaved) compared to fibrinogen levels of at least about 0.3 to 0.6 mg/ml sample or no fibrinogen at all in the sample, leading to the steepest curve (see FIG. 1). The rate of signal generation is directly dependent on fibrinogen concentration in the sample and is an indicator of the competition between enzymatic cleavage of fibrinogen and enzymatic cleavage of the (artificial and/or natural)-substrate containing detectable moiety, both reactions being catalyzed by the activity of the serine endopeptidase as defined herein.

A suitable sample to be used for the performance of the present invention might be any liquid containing an unknown concentration of fibrinogen, in particular blood or plasma, preferably isolated from mammals, such as e.g. either isolated from human or animals, such as e.g. cattle, horse or common house pets. In case of a blood sample, the blood might be freshly taken from the patient/test object in form of a whole (venous or arterial) blood capillary sample which might be collected in a vacutainer or from finger puncture (i.e. un-processed blood sample). The sample might be furthermore processed in any other form, including the use of frozen samples (i.e. processed blood sample). The method as described herein is also applicable to plasma samples, in either un-processed or processed form, such as e.g. frozen, separated and the like.

In one embodiment, the present invention is directed to a method for measuring the fibrinogen level in a sample as defined herein, said sample being selected from whole fresh blood, as well as a diagnostic kit used for such measurement, wherein the measurement is preferably in the presence of an amperogenic or chromogenic (detection) substrate.

In one embodiment, the present invention is directed to a method for measuring the fibrinogen level in a sample as defined herein, said sample being selected from plasma, as well as in a diagnostic kit used for such measurement, wherein the measurement is preferably in the presence of a chromogenic or amperogenic (detection) substrate.

Some advantageous features of the method for measuring the fibrinogen level as well as a diagnostic kit used for such measurement as defined herein compared to the typical laboratory testing known so far are as follows:

(1) Simple use compared to e.g. regular PT

(2) No need for the generation of a calibration curve, i.e. no reference material e.g. fibrinogen standards are needed

(3) Quick results at low cost

(4) Much more enhanced patient service, i.e. quicker and more accurate measurement of fibrinogen level with proper medical or surgical interventions to be decided much faster and more reliably

(5) Reduced risks and costs in transport and processing of the samples

(6) No interference with anti-coagulants such as e.g. heparin, NOACs and/or DOACs

(7) Targeting of a (preferably) single enzymatic step compared to assays relying on multi-step cascades

(8) Regarding devices with only INR functionality, such as e.g. the i-STAT®, the PT-INR results are much more reliable when factoring in the fibrinogen level of each patient.

In one aspect, the present invention is directed to a method for measuring the fibrinogen level in a sample as well as a diagnostic kit used for such measurement, comprising the following steps:

(1) providing a sample, e.g. blood taken from a patient or test object, such as e.g. human or animal blood taken from finger puncture, venous or arterial blood from a vacutainer or plasma;

(2) introduction of the sample into the respective cartridge or test-stripe (depending on the detection system or device) comprising all necessary components, such as e.g. serine endopeptidase, detection-substrate linked to detectable moiety, optionally inhibitors, physical channels, and detector or part of detector found in the device;

(3) introduction of the cartridge or test-stripe into the respective device; and

(4) analysis of the sample including reporting and transmitting of the result, i.e. the fibrinogen level in the sample.

The method and/or diagnostic kit according to the present invention can be performed on various known test devices, such as any known lab-based coagulation analyzer including but not limited to the ones specified above.

Depending on the test system/device, the novel method and/or diagnostic kit can be used with either wet or dry chemistry, i.e. wherein the components (including the substrate, enzyme, inhibitors and the like) are in a liquid form, as e.g. in a tube/cartridge or wherein the components (substrate, enzyme, inhibitors and the like) are in solid form as e.g. on a test strip. The sample to be measured, e.g. blood or plasma sample, is brought into contact with said components and the respective signals are measured by the device of choice. The test device might be connected to a remote device such as a tablet computer or smart phone.

In one embodiment, the measurement of fibrinogen level as defined herein as well as a diagnostic kit used for such measurement is performed in a processed or un-processed sample, particularly blood or plasma sample, preferably human blood or plasma sample, using CoaguChek® system from Roche Diagnostics, wherein a substrate including but not limited to substrates selected from the group consisting of a substrate according to formula (I) to (X) listed in Table 1, substrates of formula (I) to (X) but with alternative protecting groups at the N-terminal part and/or substrates of formula (I) to (X) with additional amino acids introduced between the protecting group and the 1^{st} N-terminal amino acid shown in formula (I) to (X), with the proviso that pNA is replaced by another detectable moiety suitable for the CoaguChek® system, such as e.g. phenylenediamine, e.g. commercially available as Electrozyme TH. Said substrate is preferably incubated together with the serine endopeptidase as defined herein, in particular batroxobin, leading to an electrochemical (or other signal depending on the detection moiety) signal measured/analyzed by the device, such as CoaguChek® device. The measured rate of signal generation is inversely proportional to fibrinogen concentration in the tested sample.

In a further embodiment, the measurement of fibrinogen level as defined herein as well as a diagnostic kit used for such measurement is performed in a sample including but not limited to processed or un-processed samples, particularly blood or plasma sample, preferably human blood or plasma sample, using i-STAT® from Axonlab/Abbott, wherein a substrate such as e.g. H-D-phenylalanyl-pipecolyl-arginine-p-amino-p-methoxydiphenylamine (PPAAM) or a substrate including but not limited to substrates selected from the group consisting of a substrate according to formula (I) to (X) listed in Table 1, substrates of formula (I) to (X) but with alternative protecting groups at the N-terminal part and/or substrates of formula (I) to (X) with additional amino acids introduced between the protecting group and the 1st N-terminal amino acid shown in formula (I) to (X), with the proviso that pNA is replaced by another detectable moiety suitable for the i-STAT® device, such as e.g. p-methoxydiphenylamine. Said substrate is preferably incubated together with the serine endopeptidase as defined herein, in particular batroxobin, leading to an electrochemical signal measured/analyzed by the suitable device, such as e.g. i-STAT® device. The measured signal is inversely proportional to fibrinogen concentration in the tested sample. The calculation of the results can be linear or non-linear between the signals and the fibrinogen concentrations (see FIG. 1).

To be performed on other devices known in the field, the measurement of the fibrinogen level as defined herein including a diagnostic kit used for measurement of the fibrinogen level as defined herein might be furthermore adapted to the respective device as known to the skilled person.

In one aspect of the present invention, the fibrinogen level of a patient or test object is measured in an emergency situation, i.e. the results should be available as fast as possible. Depending on the device detection system, the fibrinogen level in a sample can be measured within about less than 10 min, such as about 7, 5, 4, 3 or even about 2 minutes.

Thus, the present invention is directed to a method for measuring the fibrinogen level in a sample as defined herein, wherein the result, i.e. the level of fibrinogen present in the sample, is available within about less than 10 min, such as about 7, 5, 4, 3 or even about 2 minutes counted from the initiation of the proteolytic cleavage of the (detection) substrate as described herein.

Direct fibrinogen measurement according to the present invention can be combined with other coagulation tests, such as including but not limited to clotting time, thrombin or antithrombin activity, tissue factor assay.

As used herein, the term “analysis” in connection with measurement of fibrinogen level in a sample as described herein includes the performance of a specific algorithm depending on the device and the (detection-)substrate, which might be a natural or an artificial substrate, particularly an artificial substrate, wherein the reaction speed of the serine endopeptidase is measured which directly correlates with the fibrinogen concentration in the sample. The terms “substrate” and “detection-substrate” are used interchangeably herein.

The terms “Batroxobin moojeni” or “batroxobin” or “reptilase” or “defibrase” are used interchangeably herein and define a serine protease isolated from Bothrops venom, in particular from B. moojeni.

As used herein, the term “snake venom serine endopeptidase” means an enzyme which is directly isolated out of the animals but also an enzyme which is synthetically synthesized based on the (sequence) information of the natural enzyme, including enzymes which are produced by fermentation or cell culture leading to recombinant enzymes with at least 55% identity to batroxobin (UniProtKB—P04971) and which are able to cleave fibrinogen as described herein.

As used herein, the terms “patient” and “test object”, which are used interchangeably herein, mean a subject (either human or animal) for which the fibrinogen level according to the present invention is measured. Thus, it includes both healthy and non-healthy subjects in the commonly used sense.

As used herein, the term “high fibrinogen level” means a concentration of about at least 5 g fibrinogen in 1 l sample, such as e.g. (human) blood or plasma. The term “low fibrinogen level” as used herein means a concentration in the range of about 0.3 to 0.6 g fibrinogen in 1 l sample, such as e.g. (human) blood or plasma.

The term “enzymatic speed” as used herein means the amount of enzymatic (cleavage) product or detectable moiety generated per unit time, such as the “v” in the Michaelis-Menten equation.

In particular, the present invention features the following embodiments:

(1) Method for direct measuring the fibrinogen level in a sample via one or more enzymatic step(s) comprising catalytical cleavage of a detection-substrate, preferably an artificial detection substrate, by a snake venom serine endopeptidase.

(2) Method for direct measuring the fibrinogen level in a sample comprising the step of catalytical cleavage of a detection substrate by the action of a snake venom serine endopeptidase, wherein the fibrinogen level is inversely proportional to the signal measured from catalytical cleavage of said detection-substrate.

(3) Method as above and as defined herein, wherein measurement of the fibrinogen level is without interference by anti-coagulants present in the sample, preferably a human or animal sample, more preferably blood or plasma sample.

(4) Method as above and as defined herein, wherein the detection-substrate is linked to a detectable moiety capable of being detected (photo)electrochemically, amperogenically, chromogenically and/or fluorogenically after being cleaved-off during the measurement.

(5) Method as above and as defined herein, wherein the snake venom serine endopeptidase is originated from snake venom of Bothrops, Agkistrodon, Echis, Protobothrops, Calloselasma, Trimeresurus or Crotalus, preferably selected from the group consisting of Bothrops moojeni, Bothrops atrox, Bothrops jararaca, Echis pyramidum, Echis carinatus, Agkistrodon rhodostoma, Protobothrops mucrosquamatus, Crotalus rhodostoma, and Crotalus adamanteus.

(6) Diagnostic assay for measurement of the fibrinogen level in a sample, comprising a snake venom serine endopeptidase together with an artificial detection-substrate catalytically cleaved by said endopeptidase, preferably by using a method as above or as defined herein.

(7) Method/assay as above or as defined herein which is used in centralized haematological or clinical laboratories, emergency rooms, emergency situations occurring even outside hospitals, medical practices, private home, paddocks, barns, or point-of-care testing (POCT) environment.

(8) Use of a snake venom serine endopeptidase in the determination of the fibrinogen level in a sample, wherein the level of fibrinogen in said sample is inversely proportional to the enzymatic speed of catalyzing an artificial substrate.

(9) Device used for measuring the fibrinogen level in a sample, preferably from human or animal, more preferably blood or plasma, wherein cleavage of a fluorogenic, chromogenic or amperogenic detection moiety linked to a substrate catalyzed by the action of a snake venom serine endopeptidase can be detected by a sensor, said detection signal being inversely proportional to the fibrinogen level in the sample.

The following examples are illustrative only and are not intended to limit the scope of the invention in any way. The contents of all references, patent applications, patents and published patent applications, cited throughout this application are hereby incorporated by reference, in particular the substrates disclosed in WO2009053834, WO2000050446 or WO2016049506.

### FIGURES

FIG. 1. The relationship between the fibrinogen levels and their signals (here as an e.g. absorption at 405 nm indicated on the y-axis) is shown in dependence of the time in sec (x-axis). The plain line indicates high concentration of fibrinogen, the dotted line indicates low concentration of fibrinogen and the dashed line indicates zero fibrinogen in the sample. For more explanation see text.

FIG. 2. The relationship between the fibrinogen level (given in g/L on the x-axis) is inversely proportional to the electrical signal generated by methoxydiphenylamine (y-axis) when using the i-STAT® system.

FIG. 3. The relationship between the fibrinogen level (given in g/L on the x-axis) is inversely proportional to the electrical signal generated by phenylenediamine (y-axis) when using the CoaguChek® system.

FIG. 4. Modeling the enzymatic kinetics of human plasma fibrinogen in either 0, 20% or 40% commercially available human plasma with Pefachrom®TH as artificial substrate and batroxobin as enzyme. The Kₘ of Pefachrom®TH-batroxobin was increased about 2-fold and more than 5-fold in the presence of fibrinogen at 0.67 and 1.35 g/L, respectively, while holding the Vₘₐₓ at similar speed. The substrate concentration is given on the x-axis, the enzyme activity is given on the y-axis. For more explanation, see text.

FIG. 5. Modeling the enzymatic kinetics of human plasma fibrinogen in either 0, 30% or 60% commercially available human plasma with Pefachrom®TH as artificial substrate and batroxobin as enzyme. The Kₘ of Pefachrom®TH-batroxobin was increased about 2.5-fold and more than 5-fold in the presence of fibrinogen at 0.8 and 1.56 g/L, respectively, while holding the Vₘₐₓ at similar speed. The substrate concentration is given on the x-axis, the enzyme activity is given on the y-axis. For more explanation, see text.

FIG. 6. Determination of fibrinogen levels in defined samples. FIG. 6A shows the pNA-release curves at different plasma Citrol-1 (PL) concentrations. PL was reconstituted and diluted to the indicated concentrations of 1.6-150%, with theoretical fibrinogen (Fg) concentrations of 0.04-3.75 g/L in the reaction carried out at room temperature in the presence of batroxobin, Pefachrom TH and Pefabloc FG. The recorded OD 405 values by a plate reader (Clariostar, BMG Labtech) at each minute were normalized against the initial background OD 405 values. The averages of the OD 405 normalized values are plotted at Y-axis, with error bars of standard deviation from 3 samples, while X-axis shows the recording time of up to 10 minutes. Each curve representing different fibrinogen concentration (represented by different shape and shade, see figure legend for details) is plotted and linked with a straight line between each recording. FIG. 6B shows the typical standard curves depicting the relationship between fibrinogen concentration and OD 405 normalized at different recording time. The X-axis is the calculated fibrinogen concentrations in the reaction, while Y-axis the OD 405 normalized from 3 replicates. Each curve represents the fibrinogen concentration-signal relationship at different recording time, e.g. 4, 6 7, 10 and 15 minutes (legend). The regression lines (solid lines) and their 95% confidence areas (contained within the dotted lines between the solid lines) at different recording time were generated by GraphPad Prism 7. FIG. 6C shows the pNA-release curves at different plasma PL concentrations and 2 other commercially available control plasmas, Control plasma P and Low abnormal control assayed plasma (Low PL). All plasmas were reconstituted according the instructions to 100% plasmas. PL was serially diluted to create standard curve spanning fibrinogen concentrations of 0.08-1.25 g/L in the reaction, for clarity, only 3.1% and 50% dilutions are plotted. Two other plasmas, Control plasma P (Siemens) and Low abnormal control assayed plasma (IL), were included in the same experimental run in which the reaction was carried out at room temperature in the presence of batroxobin, Pefachrom TH and Pefabloc FG. The recorded OD 405 values at each minute were normalized against the initial background OD 405 values. The averages of the OD 405 normalized values are plotted at Y-axis, with error bars of standard deviation from 3 samples, while X-axis shows the recording time of up to 10 minutes. Each curve representing different fibrinogen concentration (represented by different shape and shade, see figure legend for details) is plotted and linked with a straight line between each recording. FIG. 6D shows the standard curve depicting the relationship between fibrinogen concentration and OD 405 normalized at the recording time at the 10th minute. The X-axis is the calculated fibrinogen concentrations in the reaction using PL, while Y-axis the OD 405 normalized from 3 replicates. The solid regression line was generated by GraphPad Prism 7. To estimate the fibrinogen concentrations of the Control plasma P and Low PL, the OD 405 normalized values of the 2 plasmas were interpolated (dotted lines with arrows), hence giving the conversion of OD signals to fibrinogen concentrations when the plasmas were at 50% concentration in the reaction. FIG. 6E shows the estimated fibrinogen concentrations of Control plasma P (Siemens) and Low abnormal control assayed plasma (HemosIL) at different time points. Y-axis denotes the fibrinogen concentrations of these 2 plasmas, Control plasma P (filled circle) and Low abnormal control assayed plasma (open circle) when they are undiluted, with error bar representing the standard deviation. The X-axis is the recording time of up to 10 minutes of the reaction explained in FIGS. 6c and 6d. The shaded areas within the dotted lines represent the 95% confidence intervals of these 2 plasmas, Control plasma P shaded by dots and Low abnormal control assayed plasma shaded by hatching lines. For more explanation, see text.

FIG. 7. Interference with anti-coagulation drugs in PT and aPTT were tested. FIG. 7A shows the Michaelis-Menten enzyme kinetics plot of batroxobin-Pefachrome TH reaction, in the presence of different concentrations of cOmplete™ protease inhibitor cocktail from Roche. The X-axis represents the increasing concentration of Pefachrome TH, while Y-axis represents the enzyme activity of batroxobin in room temperature. The curves represent the relationships of the enzyme activities in the absence (control) and 0.03x-2x of recommended usage concentrations of cOmplete™ protease inhibitor cocktail from Roche. Increasing usage of the protease inhibitor cocktail suppressed the enzyme activity of batroxobin. The suppression was of mixture of types of inhibitions, where Vmax was reduced and Km was increased. FIG. 7B a 7C show the Michaelis-Menten constance (Km) (FIG. 7B) and Vmax (FIG. 7C) of batroxobin-Pefachrome TH substrate, in the presence of different concentrations of cOmplete™ protease inhibitor cocktail from Roche, as shown in FIG. 7a. The parameters were estimated by GraphPad Prism 7. The Km was increased when the concentration of the inhibitor cocktail was increased in the batroxobin-Pefachrome TH substrate reaction, while the reverse was true for Vmax. Error bar is representing the 95% confidence interval around the average value of the Km or Vmax, while the error bars of those values obtained from the reaction performed in higher inhibitor concentrations were omitted due to the extremely large confidence interval. The batroxobin-Pefachrome TH substrate reaction was affected by a cocktail of general protease inhibitors, but not by the typical therapeutic and non-therapeutic inhibitors in blood coagulation. For more explanation, see text.

FIG. 8. Interference studies with different DOACs using the inventive batroxobin-Pefachrome TH enzymatic reaction. FIG. 8A shows the Michaelis-Menten enzyme kinetics plot of batroxobin-Pefachrom TH reaction in the presence of different concentrations of Dabigatran. The X-axis represents the increasing concentration of Pefachrome TH, while Y-axis represents the enzyme activity of batroxobin in room temperature. The curves represent the relationships of the enzyme activities in the presence of 0 ng/mL (as negative control) and 31-500 ng/mL of Dabigatran. Increasing usage of the Dabigatran did not significantly suppress the enzyme activity of batroxobin. FIG. 8B shows the Michaelis-Menten enzyme kinetics plot of batroxobin-Pefachrom TH reaction in the presence of different concentrations of 0.13-2.0 μg/mL Argatroban. Testing was performed as in FIG. 8A. FIG. 8C shows the Michaelis-Menten enzyme kinetics plot of batroxobin-Pefachrom TH reaction in the presence of different concentrations of 38-600 ng/mL Rivaroxaban. Testing was performed as in FIG. 8A. FIG. 8C shows the Michaelis-Menten enzyme kinetics plot of batroxobin-Pefachrom TH reaction in the presence of different concentrations of Dabigatran, Argatroban and Rivaroxaban. The X-axis represents the increasing concentration of Pefachrome TH, while Y-axis represents the enzyme activity of batroxobin in room temperature. For more detail plot of each drug treatment, please refer to the individual graph (8a: Dabigatran, 8b: Argatroban, 8c: Rivaroxaban). Increasing usage of the drugs did not significantly suppress the enzyme activity of batroxobin. FIG. 8D shows Michaelis-Menten constant (Km), FIG. 8E shows Vmax of batroxobin-Pefachrome TH substrate, in the presence of different concentrations of Dabigatran, Argatroban or Rivaroxaban, as shown in FIG. 8a-8d. The Km was not significantly affected by all concentrations of all inhibitors. Since Km of batroxobin-Pefachrome TH substrate reaction was much more influenced by the presence of fibrinogen, this fibrinogen test principle should be well resistant to the presence of DM and DXaIs. The presence of very high doses of drug could reduce slightly Vmax, but the effect on the fibrinogen assay can be considered negligible. Error bar is representing the 95% confidence interval around the average value of the Km or Vmax. For more explanation, see text.

FIG. 9. Interference with chemicals is shown. FIG. 9A shows the Michaelis-Menten enzyme kinetics plot of batroxobin-Pefachrom TH reaction in the presence of chemicals known to inhibit coagulation and fibrinolysis pathways. The X-axis represents the increasing concentration of Pefachrome TH, while Y-axis represents the enzyme activity of batroxobin in room temperature. The curves represent the relationships of the enzyme activities in the presence of 6 U/mL of Fragmin (a low molecular weight heparin available from Pfizer), as well as combined 10 TIU/mL aprotinin and 0.1 M 6-aminocaproic acid. Comparing to the untreated reaction (control), these agents did not significantly influence the activity. FIG. 9B shows Michaelis-Menten constant (Km), FIG. 9C shows Vmax of batroxobin-Pefachrome TH substrate in the presence of chemicals known to inhibit coagulation and fibrinolysis pathways. The Km was not significantly affected by all concentrations of all inhibitors. Since Km of batroxobin-Pefachrome TH substrate reaction was much more influenced by the presence of fibrinogen, this fibrinogen test principle should be well resistant to the presence of these inhibitors. Error bar is representing the 95% confidence interval around the average value of the Km or Vmax. For more explanation, see text.

FIG. 10. The pNA-release curves at 2 different PL concentrations to demonstrate the adaptability of the batroxobin-Pefachrome TH reaction. PL was diluted to 4% and 24% to create reactions with fibrinogen concentrations of roughly 0.1 and 0.7 g/L, respectively, in the reaction run at 37° C. in the appropriate amount of batroxobin and Pefachrome TH (refer to the legend for details). The recorded OD 405 values at each minute (X-axis) were normalized against the initial background OD 405 values, hence the OD 405 normalized as Y-axis. The averages of the OD 405 normalized values are plotted at Y-axis, with error bars of standard deviation from 3 samples, while X-axis shows the recording time of up to 10 minutes. Each curve represented by straight line linking the averages of the OD 405 normalized values depicts the reaction speed at different conditions (refer to the legend for details).

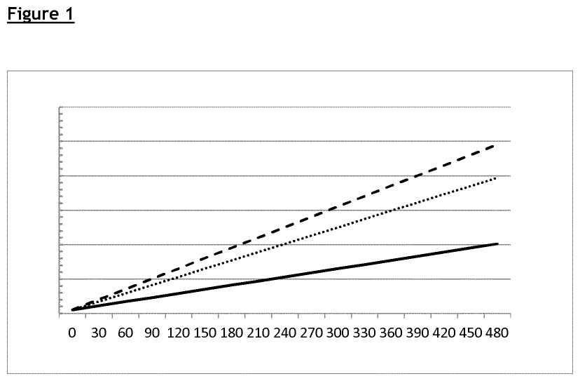

### EXAMPLES

### Example 1: Whole Blood Fibrinogen Level Measurement on PoC Device with PT-INR and ACT Functionalities

Fibrinogen level measurement in a sample using the i-STAT® point of care system (Axonlab/Abbott) is described herein, which should enable the user of the device to determine the fibrinogen level from the test object (patient) within a very short time.

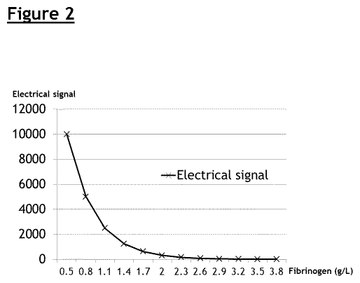

The test should work very similar to the existing prothrombin time (PT) offered by i-STAT®, except giving INR information triggered by tissue factor. The new fibrinogen utilizes the snake venom protein, batroxobin, to convert fibrinogen into fibrin. In the presence of an artificial detection-substrate including PPAAM or Pefachrome®TH for thrombin-like serine protease, and batroxobin, said artificial substrate is competing with the fibrinogen. With fixed amounts of batroxobin and PPAAM in the test, the relationship of fibrinogen concentration and of the electrochemical signal generated by the amount of the detection-substrate PPAAM can be determined. The fibrinogen is competing with PPAAM for batroxobin, resulting in a relationship between fibrinogen levels and electrochemical signals which are inversely proportional (FIG. 2).

### Example 2: Fibrinogen Assay with CoaguChek® from Roche Diagnostics

Fibrinogen level measurement in a sample using the CoaguChek® XS point of care device from Roche Diagnostics GmbH is described herein, which should enable the user of the device to determine the fibrinogen level in the whole blood sample from the test object (patient) within a very short time.

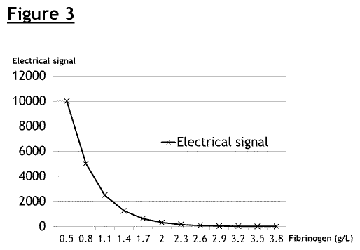

The test should work very similar to the existing prothrombin time (PT) test offered by CoaguChek® XS. The new fibrinogen test utilizes the snake venom protein, batroxobin, to convert sample fibrinogen into fibrin. In the presence of an artificial detection substrate including Electrozyme TH or Pefachrome®TH, i.e. substrates for thrombin-like serine protease, and batroxobin said artificial substrate is competing with the fibrinogen. With fixed amounts of batroxobin and the detection-substrate in the test, the relationship of fibrinogen concentration and electrochemical signal generated by the amount of active detection-substrate can be determined. Since the fibrinogen is competing with the detection-substrate for batroxobin, the relationship between fibrinogen levels and electrochemical signals are inversely proportional to each other (FIG. 3).

### Example 3: Fibrinogen Assay with a Standard Spectrophotometer

Measurement of the fibrinogen level using Pefachrom®TH containing the detectable moiety para-nitroaniline (pNA) and batroxobin together with various human plasma concentrations was used with CLARIOstar® (BMG Labtech). Again, competition between fibrinogen as natural substrate and the artificial substrate Pefachrome®TH for cleavage by the enzyme batroxobin (E) was measured.

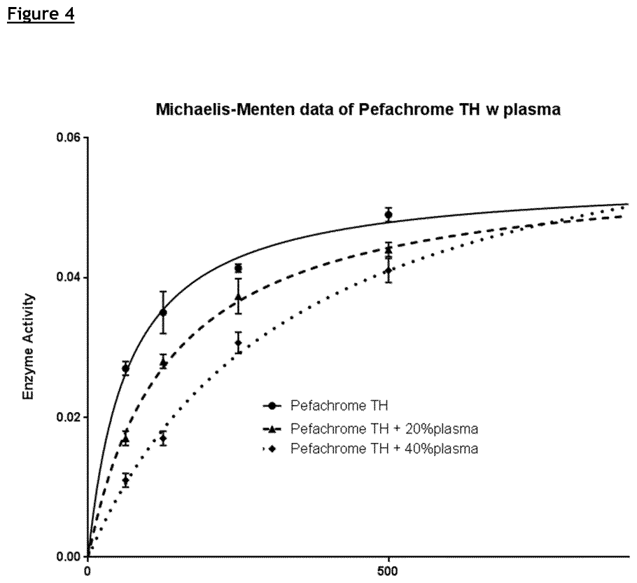

Commercially available human plasma in different dilutions, 30% and 60%, was used as source of fibrinogen. Samples of different fibrinogen levels, 0.8 and 1.56 g/L, respectively, were prepared. The enzymatic activity was calculated based on the amount of pNA released per minute. The amount of pNA is directly proportional to the absorption at 405 nm. Different concentrations of Pefachrom®TH and fibrinogen were tested in the presence of (E) in terms of the velocity of pNA release, the results and analysis are summarized in the FIG. 4 and Table 2.

Based on the Michaelis-Menten enzyme kinetic modeling, the presence of fibrinogen was altering the Michaelis-Menten constants (Kₘ) of the enzyme-substrate reactions, while the maximum enzymatic reaction speed (Vₘₐₓ) of the reactions were not significantly altered. The increased Kₘ and similar Vₘₐₓ consistently demonstrated the non-inhibitory competition between the fibrinogen and Pefachrom®TH.

Based on the Michaelis-Menten equation, wherein K_{cat}, which is almost constant in this case, denotes the maximum number of substrate molecules per active site per second, and the concentrations of both (S) and (E) are the same too in the reactions, the increased in Kₘ significantly affects the enzymatic reaction speed (v). The change of the enzymatic reaction speed, due to the presence of fibrinogen, can easily be measured and provides the estimation of fibrinogen concentration. Using Pefachrom®TH as the artificial substrate (S), we monitored the v of the pNA generation by batroxobin as enzyme (E) in the presence of different levels of human plasma derived fibrinogen, as shown in Table 2. The changes in v were due to the presence of fibrinogen, and the decrease in v was directly proportional to the increase in fibrinogen concentration, which was due to the increase in Kₘ based on the Michaelis-Menten enzyme kinetics.

TABLE 2
The enzymatic reaction of Pefachrom ®TH via cleavage
by batroxobin in the presence of different concentrations
of human plasma-derived fibrinogen. The enzymatic reaction
time after 7 or 10 min was measured at 405 nm. The data is
based on 2 independent measurements. For more details see text.

| Plasma | Fibrinogen | OD405 | OD405 |
|---|---|---|---|
| conc. [%] | [g/l] | (7 min) | (10 min) |
| 0 | 0 | 0.1175 | 0.158 |
| 20 | 0.674 | 0.095 | 0.132 |
| 40 | 1.348 | 0.063 | 0.085 |

Continuing with Pefachrom®TH as the artificial substrate (S) here, we monitored the v of the pNA generation by batroxobin in the presence of different levels of human plasma derived fibrinogen (Table 2). The changes in v were due to the presence of fibrinogen, and the decrease in v was inversely proportional to the increase in fibrinogen concentration, which was due to the increase in Kₘ based on the Michaelis-Menten enzyme kinetics.

### Example 4: Fibrinogen Assay with a Standard Spectrophotometer

Measurement of the fibrinogen level using Pefachrom®TH containing the detectable moiety para-nitroaniline (pNA) and batroxobin together with various human plasma concentrations was used with CLARIOstar® (BMG Labtech). Again, competition between fibrinogen as natural substrate and the artificial substrate Pefachrome®TH for cleavage by the enzyme batroxobin (E) was measured.

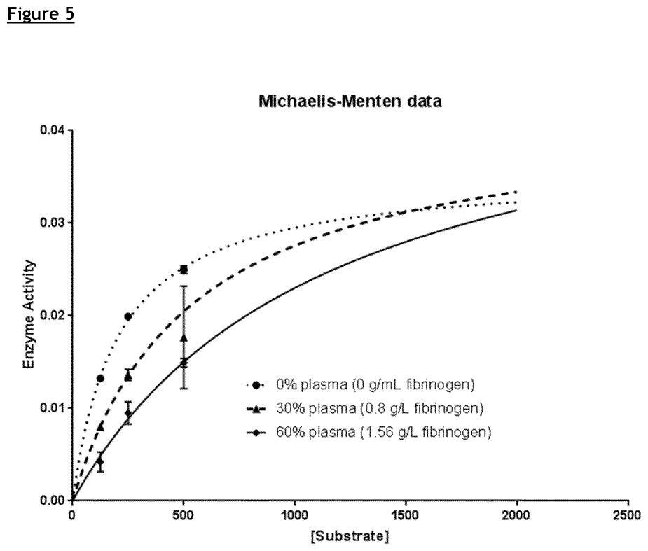

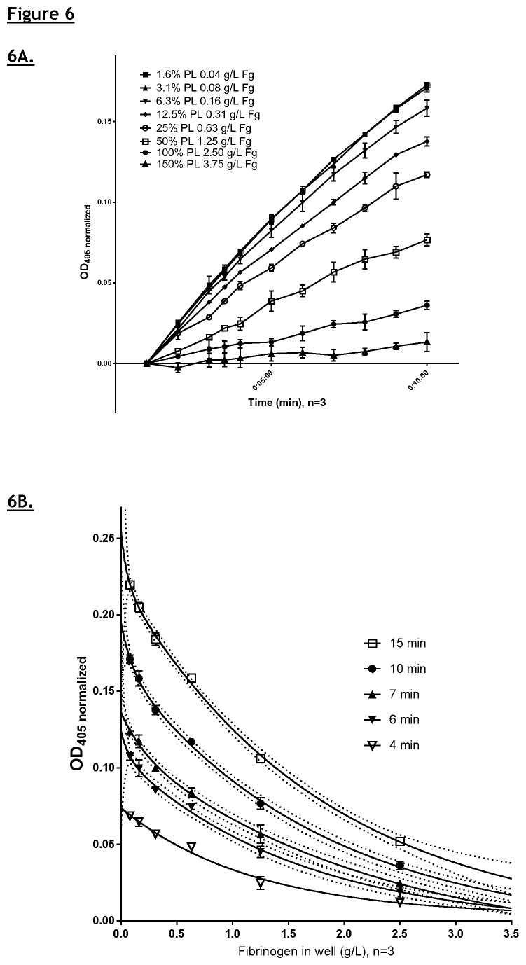

Commercially available human plasma in different dilutions, 30% and 60%, was used as source of fibrinogen. Samples of different fibrinogen levels, 0.8 and 1.56 g/L, respectively, were prepared. The enzymatic activity was calculated based on the amount of pNA released per minute. The amount of pNA is directly proportional to the absorption at 405 nm. Different concentrations of Pefachrom®TH and fibrinogen were tested in the presence of (E) in terms of the velocity of pNA release, the results and analysis are summarized in the FIG. 5 and Table 3.

Based on the Michaelis-Menten enzyme kinetic modeling, the presence of fibrinogen was altering the Michaelis-Menten constants (Kₘ) of the enzyme-substrate reactions, while the maximum enzymatic reaction speed (Vₘₐₓ) of the reactions were not significantly altered. The increased Kₘ and similar Vₘₐₓ consistently demonstrated the non-inhibitory competition between the fibrinogen and Pefachrom®TH.

Based on the Michaelis-Menten equation, wherein K_{cat}, which is almost constant in this case, denotes the maximum number of substrate molecules per active site per second, and the concentrations of both (S) and (E) are the same too in the reactions, the increased in Kₘ significantly affects the enzymatic reaction speed (v):

$$v = \frac{{k}_{cat} [ E ] [ S ]}{{K}_{m} + [ S ]}$$

The change of the enzymatic reaction speed, due to the presence of fibrinogen, can easily be measured and provides the estimation of fibrinogen concentration. Using Pefachrom®TH as the artificial substrate (S), we monitored the v of the pNA generation by batroxobin as enzyme (E) in the presence of different levels of human plasma derived fibrinogen, as shown in Table 3. The changes in v were due to the presence of fibrinogen, and the decrease in v was inversely proportional to the increase in fibrinogen concentration, which was due to the increase in Kₘ based on the Michaelis-Menten enzyme kinetics.

TABLE 3
The enzymatic reaction of Pefachrom ®TH via cleavage
by batroxobin in the presence of different concentrations of
human plasma-derived fibrinogen. The enzymatic reaction time
after 7, 11 or 16.5 min was measured at 405 nm. The data is
based on 2 independent measurements. For more details see text.

| Fibrinogen | OD405 | OD405 | OD405 |
|---|---|---|---|
| [g/l] | (7 min) | (10 min) | (16.5 min) |
| 0 | 0.159 | 0.262 | 0.394 |
| 0.8 | 0.131 | 0.209 | 0.310 |
| 1.56 | 0.062 | 0.105 | 0.161 |
| 3.1 | 0.031 | 0.050 | 0.080 |

Continuing with Pefachrom®TH as the artificial substrate (S) here, we monitored the v of the pNA generation by batroxobin in the presence of different levels of human plasma derived fibrinogen (Table 3). The changes in v were due to the presence of fibrinogen, and the decrease in v was directly proportional to the increase in fibrinogen concentration, which was due to the increase in Kₘ based on the Michaelis-Menten enzyme kinetics.

### Example 5: Plasma Fibrinogen Concentrations Determined by Batroxobin Enzyme Kinetics

Measurement of the fibrinogen level was performed using well characterized plasmas, which were commercially available, to challenge the feasibility of this innovative principle of fibrinogen measurement in blood sample.

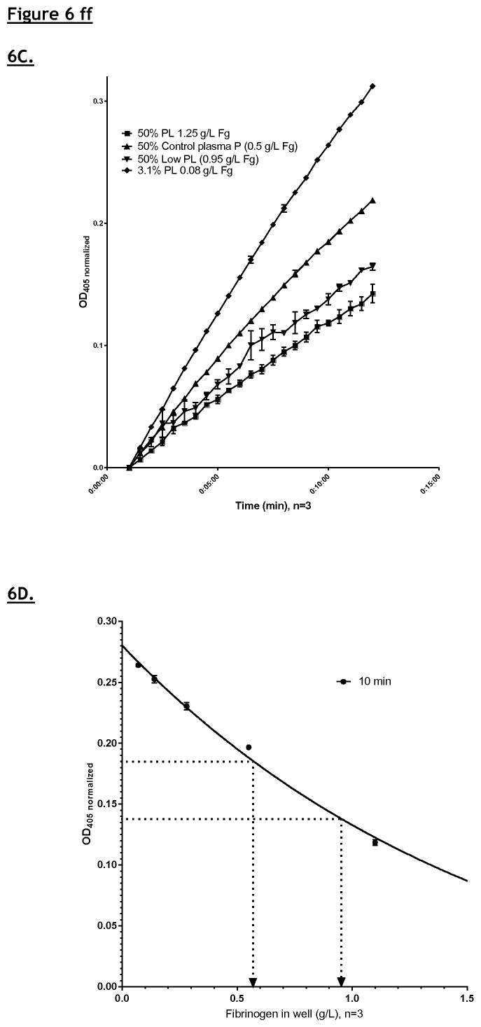

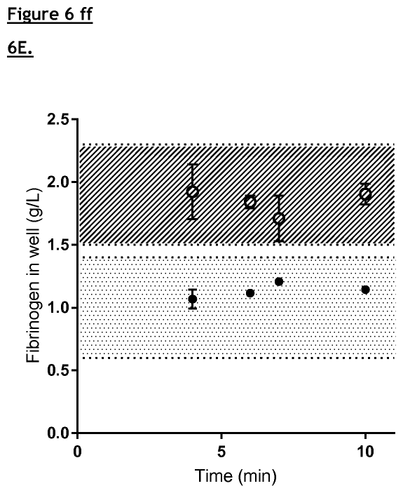

The current well accepted fibrinogen assay is clot-based Clauss test. The control plasmas, available from Siemens and Instrumentation Laboratory (IL), are used in the standard Clauss test as controls in fibrinogen measurement, and the fibrinogen concentrations were well characterized (Table 4). To test the feasibility of this chromogenic fibrinogen assay in plasma fibrinogen determination, briefly, the calibration curves was obtained from serially diluted Citrol-1, a control plasma from Siemens (FIG. 6a,6b). The other 2 plasmas with different fibrinogen levels (Table 4), Control plasma P (Siemens) and Low abnormal control assayed plasma (IL), were assayed to estimate their fibrinogen concentrations by intrapolating from the calibration curve created using Citrol-1 (see FIGS. 6c, 6d and 6e for details).

TABLE 4
Plasma samples and their fibrinogen concentrations. Control plasma P (from
Siemens), low abnormal control assayed plasma (from IL) and control plasma
Citrol-1 (from Siemens) as stated in the product inserts were extracted
and summarized in this table. Each fibrinogen concentration is displayed
in average value and confidence interval (in bracket) determined by different
instrument/analyzer and reagent. For more details see text.

| | | Fibrinogen concentration [g/l] | | |
|---|---|---|---|---|
| Analyzer | reagent | Control plasma P | HaemosiL | Citrol-1 |
| Siemens CA | Multifibren U | 1.0 (0.6-1.4) | | 2.5 (2.2-2.8 Cl) |
| systems | | | | |
| | Dade Thrombin | 0.8 (0.4-1.2) | | 2.5 (2.2-2.8 Cl) |
| | Reagent | | | |
| BCS XP | Multifibren U | 1.0 (0.6-1.4) | | 2.6 (2.3-2.9 Cl) |
| | PT-Fibrinogen | | 1.5 (1.1-1.9) | |
| ACL classic | PT-fibrinogen | | 1.8 (1.4-2.2) | |
| | HS PLUS | | | |
| | Fibrinogen-C | | 1.9 (1.5-2.3) | |
| | PT-Fibrinogen | | 1.4 (1.0-1.8) | |
| ACL TOP | PT-fibrinogen | | 1.8 (1.4-2.2) | |
| | HS PLUS | | | |
| | Fibrinogen-C | | 1.9 (1.4-2.4) | |

Based on the current conditions described in FIG. 6, the assay was able to have good differentiation or separation between fibrinogen levels of 0.05 and 0.3 g/L (FIG. 6a & 6b). The inversely proportional relationship between the fibrinogen concentration and the measurable signal as OD at 405 nm was clearly demonstrated in this clinically characterized control plasma (FIG. 6a & 6b). A calibration curved was produced and the fibrinogen concentrations of the 2 plasmas were estimated (FIG. 6c & 6d). The estimations of these 2 plasmas were matching very well with the values given by the plasma suppliers (FIG. 6e/Table 4). Hence, the clinically relevant plasma samples of abnormally low fibrinogen levels were correctly estimated using the inventive method, based on batroxobin enzyme kinetics.

### Example 6: Interference Study of Direct Thrombin (DTI) and Direct FXa Inhibitor (DXaI) in the Fibrinogen Measurement Based on Batroxobin Enzyme Kinetics

In this example the advantageous property of the inventive method has been tested against interference from direct thrombin or FXa inhibitors.

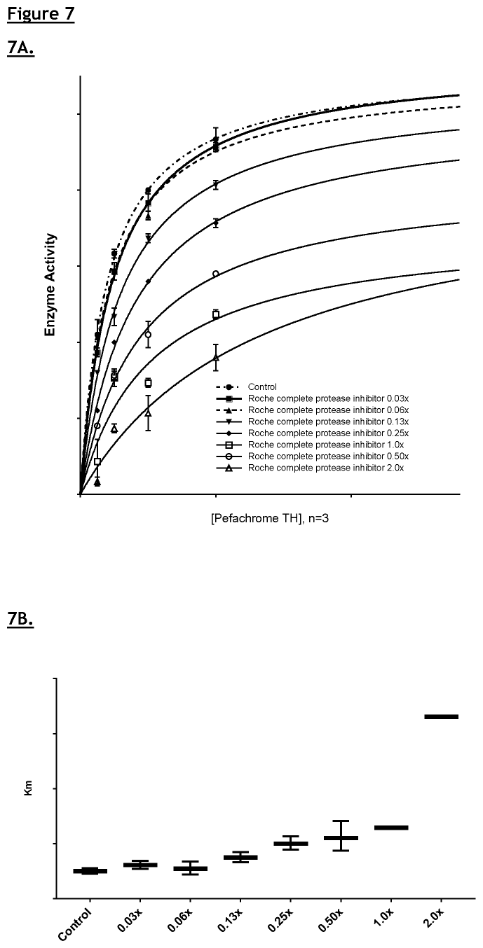

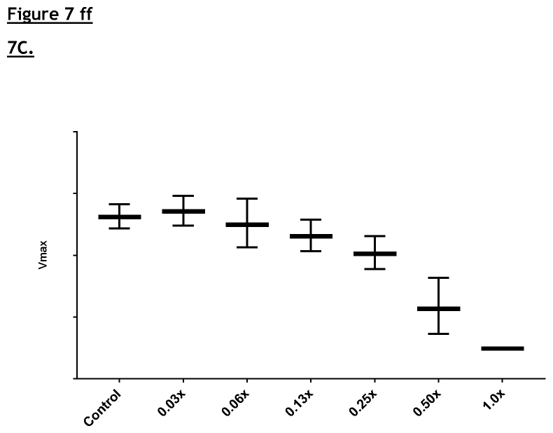

In the emergency situation when a patient needed a fibrinogen level estimation, the fibrinogen test has to be free of as many interfering factors as possible. The use of direct oral anti-coagulants (DOACs), including DM and DXaIs, are getting more common to prevent thrombosis in many diseases. In this example, we tested 3 protease inhibitors Dabigatran (a DTI), Argatroban (a DTI) and Rivaroxaban (aDXaI) in our new method to assess the interference of these representative drugs of this class in our fibrinogen measurement method. To evaluate the inhibitory effect of these pharmaceutical agents, we looked into the effects of these agents in the enzyme kinetics between batroxobin and its substrate Pefachrome TH. The batroxobin-Pefachrome TH enzymatic reactions were tested in the presence of a cocktail of protease inhibitors obtained from Roche, cOmplete™ protease inhibitor cocktail (see FIG. 7a). The enzyme kinetic parameters like Vmax and Km were estimated based on Michaelis-Menten enzyme kinetic model, and the inhibitory effects of the cocktails on the enzymatic reaction was clearly visible (FIG. 7a), and the Michaelis-Menten parameters, Vmax and Km, indicate mixtures of inhibitions (FIG. 7b & 7c).

With the establishment of enzyme kinetic study, the interference of the DM and DXaI was started by testing the potency of these drugs in the two common blood coagulation tests, namely Prothrombin Time (PT) and activated Partial Thromboplastin Time (aPTT). In the presence of these DTI and DXaI, these two tests will display delays in clotting time. Based on this principle, we assessed the potency of these drugs by applying the reported peak and trough plasma concentrations in PT and aPTT. The results are shown in Table 5: the direct thrombin and FXa inhibitors, denoted as DTI and DXaI respectively, were able to delay blood clotting time based on Prothrombin Time (PT) and activated Partial Thromboplastin Time (aPTT). The reported maximum and trough concentrations of the Dabigatran was between 447-10 ng/mL, while Rivaroxaban was 535-6 ng/mL. The control plasma was spiked individually with different amounts and kinds of inhibitors, and the clotting times of PT and aPTT were recorded by BCS-XP (Siemens).

TABLE 5
Summary of the inhibitory effects of DTIs and DXaI within the
clinical range of concentrations which were also tested on the
influence of batroxobin-mediated fibrinogen assay. They are denoted
by the name of the inhibitor along with the final concentration
in the plasma. The negative control (denoted as neg. control)
was the control plasma without any spiking of inhibitor. Another
negative control (denoted as ISTH) was the recommended control
plasma by International Society on Thrombosis and Hemostasis
(ISTH), also without any spiking of inhibitor.

| | | PT-test | | | aPTT-test | | |
|---|---|---|---|---|---|---|---|
| | | CT | Mean | | CT | Mean | |
| Drug | | (sec) | (sec) | delay | (sec) | (sec) | delay |
| Dabigatran | | 19.81 | 19.90 | 97% | 91.09 | 90.98 | 183% |
| 500 | ng/ml | 19.98 | | | 90.87 | | |
| Dabigatran | | 10.58 | 10.64 | 6% | 41.3 | 41.28 | 28% |
| 31 | ng/ml | 10.69 | | | 41.2 | | |
| Rivaroxaban | | 19.42 | 19.37 | 92% | 80.73 | 80.40 | 150% |
| 600 | ng/ml | 19.32 | | | 80.07 | | |
| Rivaroxaban | | 10.74 | 10.68 | 6% | 39.46 | 39.45 | 23% |
| 38 | ng/ml | 10.61 | | | 39.44 | | |
| Argatroban | | 12.26 | 12.25 | 22% | 55.77 | 55.62 | 73% |
| 2.0 | μg/ml | 12.24 | | | 55.46 | | |
| Argatroban | | 10.18 | 10.20 | 1% | 34.62 | 34.62 | 8% |
| 0.13 | μg/ml | 10.21 | | | 34.62 | | |

| ISTH | 9.65 | 9.68 | 28.77 | 28.71 |
|---|---|---|---|---|
| | 9.70 | | 28.64 | |
| Neg. control | 10.10 | 10.08 | 32.16 | 32.14 |
| | 10.05 | | 32.12 | |

A spectrum of potencies of strong to weak based on the modes of action and concentrations was detected, and the results were consistent with literatures.

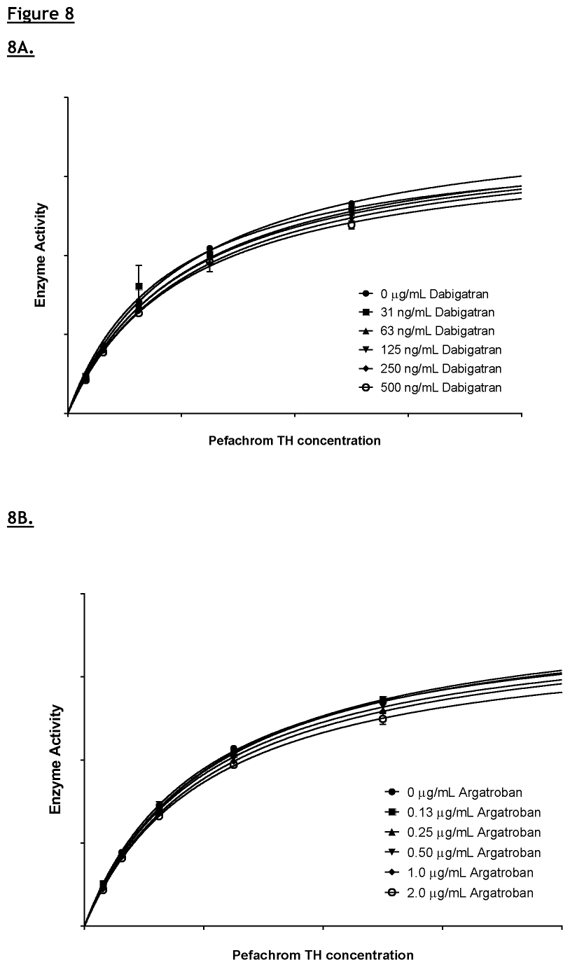

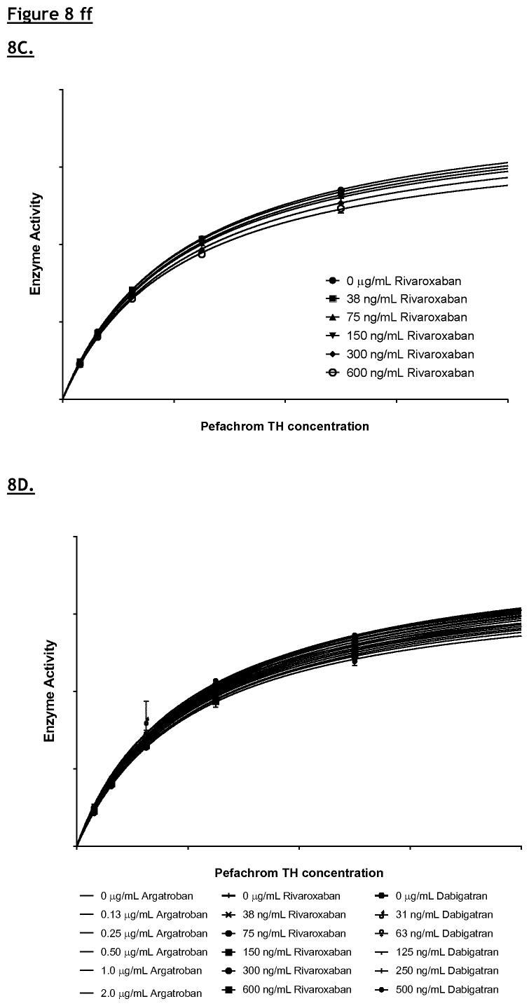

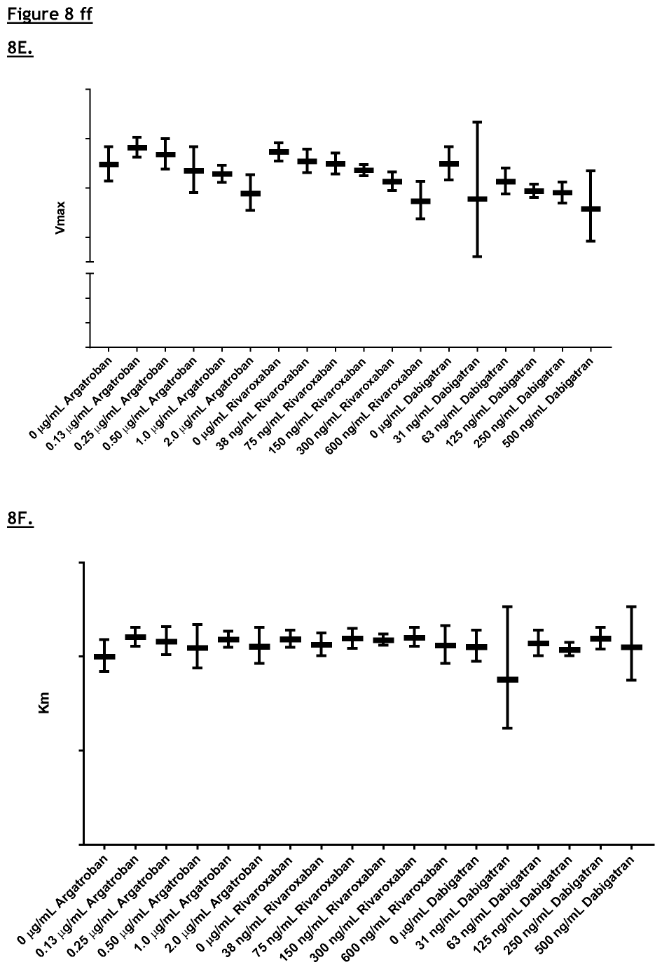

Having demonstrated the potency of the drugs in inhibiting thrombin and FXa, the interfering effects of Dabigatran, Argatroban and Rivaroxaban were studied in the batroxobin-Pefachrome TH enzymatic reaction. In the enzyme kinetic study, we included from 0-500 ng/mL of Dabigatran into the batroxobin-Pefachrome TH reaction (see FIG. 8a). The similar study was carried for the drug Argatroban between the range of 0-2 μg/mL (see FIG. 8b). Additionally, Rivaroxaban of 0-600 ng/mL was applied to this enzyme kinetic study (see FIG. 8c). In this study, we did not observe the strong inhibitory effects with DTIs and DXaI as with the cocktail of protease inhibitors from Roche (comparing FIGS. 8a to 8c). There might be very slight reduction in Vmax at the highest dose of each drug, but the Km stayed very constant throughout the different drugs and concentrations (FIG. 8e & 8f). Since Km is the main parameter being shifted according to the presence of fibrinogen, i.e. the higher the fibrinogen concentration, the larger Km is increased (refer to Example 3 & 4 for details), we can safely rule out the assay will be interfered by the typical DTIs and DXaIs or common DOACs.

### Example 7: Interference Study of Chemicals Known to Affect Clot-Based Assay

In this example the advantageous property of the inventive method was tested against chemical interference known to affect clot-based assays.

Similar to the test performed in the previous example, enzyme kinetics between batroxobin and its substrate Pefachrome TH were evaluated. Potential interfering substances, which have been demonstrated to interfere in clot-based assays, are heparins (including unfractionated and low molecular weight heparins, UFH and LMWH), hirudin, EDTA and fibrinogen degradation products (FDPs). Heparins and hirudin are therapeutic substances in the treatment of thrombosis. Increased FDPs presence in plasma is due to conditions that increase fibrinolysis and fibrinogen lysis. The normal FDP level is around 5-8 μg/mL. Higher FDP concentration is known to inhibit clot formation. Pharmaceutical substances to inhibit fibrinolysis in the treatment of hemorrhages like aprotinin and 6-aminocaproic acid were also included in this study. Additionally, a colloid hydroxyethyl starch (HES), used in the plasma expander solution, was subjected to interfering activity study.

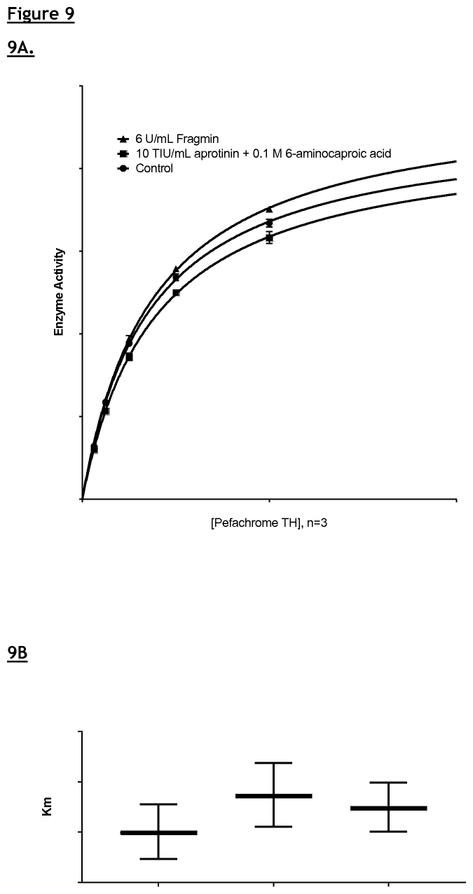

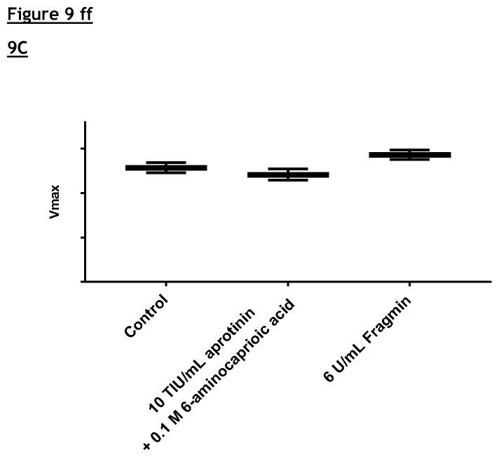

First, very high concentrations of low molecular weight heparin (Fragmin), aprotinin and 6-aminocaproic acid were tested for their interference in the batroxobin-Pefachrome TH enzyme reaction (see FIG. 9a). The predicted Vmax and Km were not significantly different from the untreated control (FIG. 9b & 9c). Hence, it is very safe to conclude that the inventive method for fibrinogen measurement will not be interfered by these substances.

We furthermore assessed the interference of unfractionated heparin (Liquemin: till 4 U/mL), calcium-chelator EDTA (till 8 mM), hirudin (till 4 U/mL), HES (till 5 mg/mL), FDP (till 57 μg/mL) using the same methodology. We failed to see significant interference coming from all these substances, indicating again the independence or non-interference of the inventive method against these substances (data not shown).

### Example 8: Adaptable Enzymatic Conditions

Since the typical PoC devices, i.e. iSTAT and CoaguCheck, warm up their blood samples to body temperature during testing, we studied this principle when operated in body temperature. We adjusted a few parameters so that we could increase the signal output and allow good differentiation at the low fibrinogen concentrations.

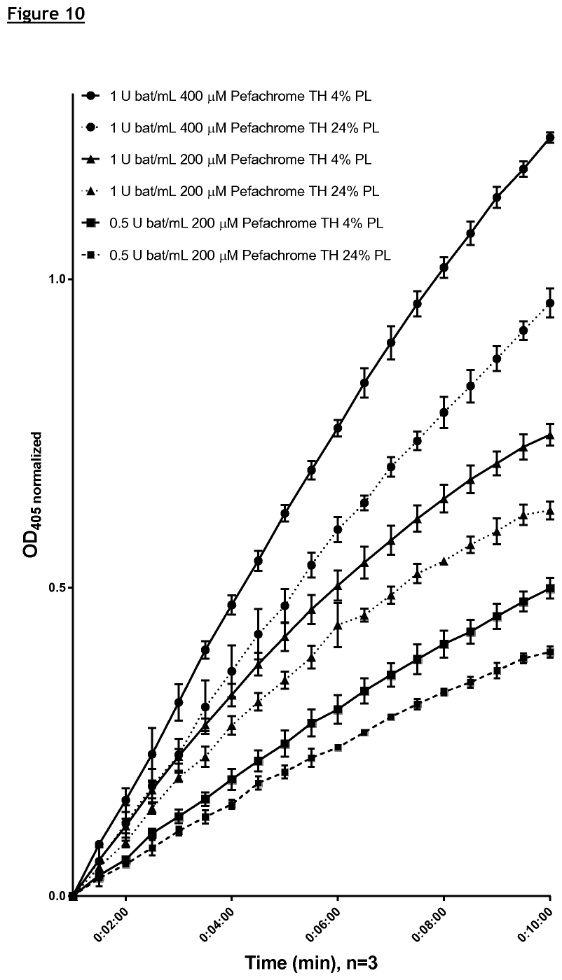

The adaptation of the inventive fibrinogen detection method based on batroxobin enzyme kinetics to body temperature was successfully performed. Parameters like the concentrations of the enzyme and substrates were adjusted to produce desirable performance at low fibrinogen concentrations in plasma (see FIG. 10).

1. A method for measuring the fibrinogen level in a sample wherein the blood coagulation cascade is inhibited, preferably inhibition of both intrinsic and extrinsic pathway.

2. A method according to claim 1 comprising an enzymatic cleavage reaction, wherein the enzymatic cleavage of fibrinogen competes with enzymatic cleavage of a detection substrate present in the sample.

3. A method according to claim 1 or comprising the use of a serine endopeptidase for enzymatic cleavage of fibrinogen.

4. A method according to claim 2, wherein the speed of enzymatic cleavage depends on the fibrinogen level in the sample.

5. A method according to claim 2 comprising measuring the proteolytic activity of a serine endopeptidase which is inversely proportional to the fibrinogen level in said sample.

6. A method according to claim 1, said method being performed in the absence of CaCl2 and/or in the absence of thrombin activity.

7. A method according to claim 1, said method comprising the presence of protease inhibitors, preferably inhibitors of fibrin polymerization, more preferably thrombin inhibitors.

8. A method according to claim 1 which does not include the generation of a calibration curve and/or the generation/presence of a fibrinogen standard.

9. A method according to claim 1, wherein the sample is selected from blood or plasma.

10. A method according to claim 1 which is used in centralized haematological or clinical laboratories, emergency rooms, emergency situations occurring even outside hospitals, medical practices, private home, paddocks, barns, or point-of-care testing (POCT) environment.

11. A diagnostic kit used for performance of a method according to claim 1.

12. A serine endopeptidase [EC 3.4.21], preferably snake venom serine endopeptidase, more preferably venombin A [EC 3.4.21.74] used in a method according to claim 1.

13. A detection substrate, preferably artificial detection substrate, particularly in combination with the serine endopeptidase according to claim 12.

14. A detectable moiety, preferably in combination with the serine endopeptidase according to claim 12.
